(19) United States
(12) Patent Application Publication
(10) Pub. No.: US 2023/0390307 A1
(43) Pub. Date: Dec. 7, 2023

(54) PEG-FREE AQUEOUS SUSPENSIONS FOR PARENTERAL ADMINISTRATION OF A CORTICOSTEROID

(71) Applicant: Pfizer Inc., New York, NY (US)

(72) Inventors: Omar Abdelrahman Salman, Ann Arbor, MI (US); Joel Aaron Bergman, Gurnee, IL (US)

(73) Assignee: Pfizer Inc., New York, NY (US)

(21) Appl. No.: 18/249,581

(22) PCT Filed: Oct. 19, 2021
(86) PCT No.: PCT/IB2021/059603

§ 371 Date: Apr. 19, 2023

Related U.S. Application Data
(60) Provisional application No. 63/104,143, filed on Oct. 22, 2020.
(60) Provisional application No. 63/252,705, filed on Oct. 6, 2021.

(51) Int. Cl. A61K 31/573; A61K 47/22; A61K 31/57; A61K 9/10

(52) U.S. Cl. CPC A61K 31/573; A61K 47/22; A61K 31/57; A61K 9/10

(57) ABSTRACT

A parenteral aqueous suspension formulation for corticosteroids without polyethylene glycol (PEG) or Polysorbate (PS) that has better resuspendability, longer stability compared to commercially available formulations, and additionally allows for stable formulations of higher concentrations of corticosteroids that were not previously feasible. Preferably, the corticosteroid is methylprednisolone acetate or medroxyprogesterone acetate.

### BACKGROUND OF THE INVENTION

Depo-Medrol® (methylprednisolone acetate) and Depo-Provera® (medroxyprogesterone acetate) are formulated as parenteral aqueous suspensions which include a vehicle containing polyethylene glycol (PEG) 3350. PEG 3350, is added to the vehicle to sterically stabilize the suspension and has been an ingredient in parenteral aqueous suspensions for over 20 years. The mechanism of stabilization is explained as follows. PEG is a nonionic water-soluble surfactant which has the chemical formula of HO(CH₂CH₂O)ₙH. Segments of PEG polymer, referred to as “anchoring” chains adsorb to the surface of Active Pharmaceutical Ingredient (API) particles to form an adsorption layer. The thickness of this layer depends on several parameters such as polymer concentration, solvency of the media, temperature, and molecular weight of the polymer. The other segments, referred to as stabilizing chains or “tails” extend into the solution [T. Tadros. Interaction forces between particles containing grafted or adsorbed polymer layers. Advances in Colloidal Interface Science, 104, 2003]. These tails interconnect to bridge between the particles, resulting in controlled flocculation. Accordingly, the API settles as loosely bridged particles which are easy to re-disperse. If no surfactant is added to the vehicle, however, the particles settle independently at a slow rate and form a compact sediment which is difficult to re-disperse [T. Tadros. Control of stability/flocculation and rheology of concentrated suspensions. Pure & Appl. Chem., 64 (11), 1992]. As shown in FIG. 1, deflocculated particles have a slow terminal velocity which is proportional to the particle diameter squared (according to Stokes law) and form a compact cake. Flocculated particles, on the other hand, settle faster to form a less packed cake, as represented by a higher sediment height, which can be easily resuspended [L. Wu; J. Zhang; W. Watanabe, Adv. Drug Delivery Rev. 63 (2011) pp. 456-469]. Note that extensive agglomeration, or uncontrolled, flocculation is undesirable since it results in very fast settling of particles, making the aspiration of accurate doses challenging and may cause plugging of a needle that is used for administration to a patient.

However, despite this benefit, there are known issues and concerns with using PEG. For example, PEG and similar surfactants such as polysorbates (PS), are known to be susceptible to autoxidation to form hydroperoxides followed by chain degradation into byproducts such as formic acid, resulting in a continuous decrease in pH until the oxygen in the headspace is depleted [M. Danbrow, E. Azaz, A. Pillersdorf. Autoxidation of polysorbates. J. Pharm. Sci., 67 (12), 1978]. In addition to pH drop, degradation of the surfactant could result in thickening of the suspension leading to content uniformity issues. To address pH drop caused by the oxidative degradation of PEG and to extend product (which comes in a vial presentation) shelf life, air in the headspace of the vial is replaced with nitrogen (nitrogen overlay), or a buffering agent is added to the formulation. However, even when using nitrogen, oxygen still degrades these products overtime. Another concern with PEG is occasional poor redispersibility (caking and a compact sediment) of the suspension after long storage periods.

There are also several publications that discuss the purported adverse safety effects of PEG on patients. Nelson discussed the risks associated with using Depo-Medrol® in intraspinal injection caused by the presence of PEG [D. A. Nelson. Dangers from methylprednisolone acetate therapy by intraspinal injection. Arch Neurol, 45, 1988]. He concluded that the use of glycols, when injected intraspinally, may result in severe effects such as sterile meningitis, arachnoiditis, or pachymeningitis. Honorio et al studied the effect of PEG on mammalian nerve impulses [T. Honorio, A. J. Gissen, G. R. Strichartz, M. J. Avaram, B. G. Covino. The effect of polyethylene glycol on mammalian nerve impulses. Anesth Analg., 66, 1987]. PEG concentration at or above 20% depressed the compound action potentials of nerve cells and decreased the conduction velocities of the A, B, and C nerve fibers. The potential toxicity of repeated topical application of antimicrobial creams containing PEG to burn patients was studied by Herold et al using an animal model (D. A. Herold. Toxicity of topical polyethylene glycol. Toxicology and Applied Pharmacology, 65, 1982). Applying this cream to open wounds in rabbits resulted in elevated total calcium, elevated osmolality gap, high anion gap metabolic acidosis, and renal failure. Absorption of PEG followed by its metabolism to nephrotoxic compounds and to mono and diacids was proposed as the cause for these symptoms. Herold et al proposed that sequential oxidation of PEG to organic acids by alcohol dehydrogenase and aldehyde dehydrogenase after absorption as the mechanism of toxicity [D. A. Herold, K. Keil, D. E. Bruns. Oxidation of polyethylene glycol by alcohol dehydrogenase. Biochemical Pharmacology, 38, 1989].

In order to address the aforementioned technical issues and purported safety concerns with PEG, a formulation of an aqueous suspension essentially free of PEG was developed, where the only excipients in the vehicle is a tonicity agent such as sodium chloride (NaCl) and a quaternary ammonium compound such as myristyl gamma picolinium chloride (MGPC). Surprisingly, it was discovered that removing PEG completely and reducing the amount of MGPC found in commercially available corticosteroid formulations resulted in unexpectedly longer-term stability compared to those commercially available formulations.

The new formulations described herein have better resuspendability and stable pH without nitrogen overlay, resulting in a much longer shelf life compared to the shelf life of Depo-Medrol® and Depo-Provera® currently available commercially.

Additionally, it was discovered that through removal of PEG, surprisingly, higher drug loading in the methylprednisolone acetate (MRA) suspension was possible by proportionally increasing the amount of MGPC. Previously, MRA concentrations above 80 mg/mL are not available commercially, in part because higher concentrations presented resuspendability challenges. By removing PEG completely, it was surprisingly discovered that stable, higher concentrations of MRA are possible despite the prior art teaching that PEG was thought to be required to stabilize the suspension. Higher concentration doses of MRA are now possible, such a single shot at 120 mg/mL of MRA, the maximum approved daily dose by the FDA. For an intramuscular injection such as this, it will help decrease pain, discomfort, etc. associated with high volume doses and multiple injections.

### SUMMARY OF THE INVENTION

The present invention provides a new pharmaceutical aqueous suspension formulation for parenteral use comprising a corticosteroid, a quaternary ammonium compound, a tonicity agent, and water, wherein the formulation is essentially free of each of polyethylene glycol and polysorbate. The new formulation does not contain PEG or (PS), which are the main cause of pH drop that results in loss of stability over time. By eliminating PEG and PS and using only a quaternary ammonium compound such as myristyl gamma picolinium chloride (MGPC) and a tonicity agent such as sodium chloride in the vehicle, the suspension has surprisingly better resuspendability and stability during its shelf life than existing commercial products.

### BRIEF DESCRIPTION OF THE DRAWINGS

FIG. 1 provides a representation of sedimentation in (a) deflocculated and (b) flocculated suspensions. Panels represent increasing time from left to right. The schematic is reproduced from L. Wu; J. Zhang; W. Watanabe, Adv. Drug Delivery Rev. 63 (2011) pp. 456-469.

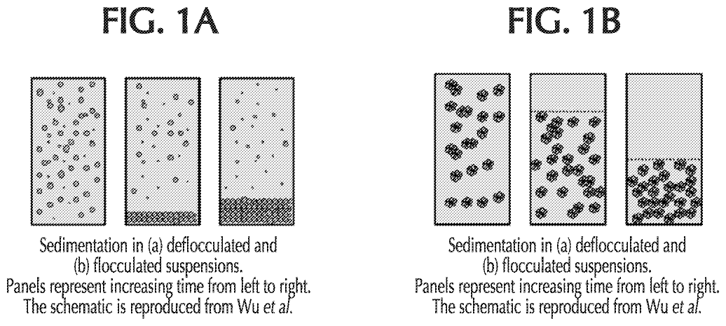

FIG. 2 provides a plot of pH drop as a function of storage time for 40 mg/mL Depo Medrol® compared with a PEG-free suspension of the invention.

FIG. 3 provides a plot of pH drop as a function of time for Depo-Provera® compared with a PEG-free suspension of the invention.

FIG. 4 provides a plot of % Settled Drug Height vs Time, i.e., sedimentation for a PEG-free 120 mg/mL MRA suspension of the invention.

FIG. 5 provides a plot of the average number of inversions (n=2, where n is number of trials) to fully resuspend a 120 mg/mL PEG-free MRA suspensions at various MGPC concentrations.

FIG. 6 provides a plot of % Settled Drug Height vs Time to show the effect of various NaCl concentrations on sedimentation of 120 mg/mL PEG-free MRA suspensions of the invention.

### DETAILED DESCRIPTION OF THE INVENTION

According to a first aspect of the invention, there is provided a pharmaceutical aqueous suspension formulation for parental use comprising a corticosteroid, a quaternary ammonium compound, an isotonicity agent, and water; and wherein the formulation is essentially free of each of polyethylene glycol and polysorbate.

In another aspect of the invention, there is provided a pharmaceutical aqueous suspension formulation for parental use comprising a corticosteroid, a quaternary ammonium compound, a tonicity agent, and water; and wherein the formulation is essentially free of each of polyethylene glycol and polysorbate.

Described below are a number of embodiments (E) of this first aspect of the invention, where for convenience E1 is identical thereto.
E1. The formulation, according to either of the two aspects of the invention, as set out just above.
E2. The formulation according to embodiment E1, wherein the quaternary ammonium compound is myristyl gamma picolinium chloride with a concentration of less than 0.5 mg/mL.
E3. The formulation according to embodiment E2, wherein the concentration of myristyl gamma picolinium chloride is less than 0.4 mg/mL.
E4. The formulation according to embodiment E3, wherein the concentration of myristyl gamma picolinium chloride is 0.07-0.3 mg/mL
E5. The formulation according to embodiment E4, wherein the concentration of myristyl gamma picolinium chloride is 0.07-0.23 mg/mL.
E6. The formulation according to any one of embodiments E1 to E5, wherein the tonicity agent is sodium chloride
E7. The formulation according to embodiment E6, further wherein the sodium chloride has a concentration of 9 mg/mL.
E8. The formulation according to embodiment E6, further wherein the sodium chloride has concentration of 10 mg/mL
E9. The formulation according to any one of embodiments E1 to E8, wherein the corticosteroid is methylprednisolone acetate.
E10. The formulation according to embodiment E9, wherein the methylprednisolone acetate has a concentration in the range of 20-160 mg/mL.
E11. The formulation according to embodiment E10, wherein the concentration of methylprednisolone acetate is in the range of 20-80 mg/mL.
E12. The formulation according to any one of embodiments E1 to E8, wherein the corticosteroid is medroxyprogesterone acetate.
E13. The formulation according to embodiment E11, wherein the medroxyprogesterone acetate has a concentration range of 135-165 mg/mL.
E14. The formulation according to embodiment E1 consisting essentially of methylprednisolone acetate or medroxyprogesterone acetate, myristyl gamma picolinium chloride with a concentration of less than 0.5 mg/mL, sodium chloride, and water.
E15. The formulation according to embodiment E1 wherein the corticosteroid is methylprednisolone acetate or medroxyprogesterone acetate, the quaternary ammonium compound is myristyl gamma picolinium chloride with a concentration of less than 0.5 mg/mL, and wherein the formulation is maintained at a pH of between 4-7 for a period of at least 100 days at 40° C.
E16. The formulation according to embodiment E15 wherein the period is at least 300 days.
E17. The formulation according to embodiment E15 wherein the period is at least 500 days.
E18. The formulation according to any one of embodiments E14 to E17, wherein the myristyl gamma picolinium chloride concentration is less than 0.4 mg/mL.
E19. The formulation according to any one of embodiments E14 to E17, wherein the myristyl gamma picolinium chloride concentration is 0.3 mg/mL or less.
E20. A vial with a headspace containing any of the formulations according to any of the embodiments E1 to E19 and E42 to E58 wherein the vial is filled with ambient air in the headspace.
E21. A method of treating allergic conditions in asthma, atopic dermatitis, contact dermatitis, drug hypersensitivity reactions, allergic rhinitis, serum sickness, or transfusion reactions in a subject; comprising administering to the subject in need of such treatment a therapeutically effective amount of the formulation according to any one of the embodiments E1 to E11 and E14 to E19.
E22. A method of treating dermatologic diseases selected from Bullous dermatitis herpetiformis, exfoliative dermatitis, mycosis fungoides, pemphigus, or severe erythema multiforme in a subject; comprising administering to the subject in need of such treatment a therapeutically effective amount of the formulation according to any one of the embodiments E1 to E11 and E14 to E19.
E23. A method treating endocrine disorders selected from adrenocortical insufficiency, congenital adrenal hyperplasia, or hypercalcemia associated with cancer, or nonsupportive thyroiditis in a subject; comprising administering to the subject in need of such treatment a therapeutically effective amount of the formulation according to one of the embodiments E1 to E11 and E14 to E19.
E24. A method of treating gastrointestinal diseases to tide the patient over a critical period of the disease in regional enteritis and ulcerative colitis in a subject; comprising administering to the subject in need of such treatment a therapeutically effective amount of the formulation according to one of the embodiments E1 to E11 and E14 to E19.
E25. A method of treating hematologic disorders selected from acquired hemolytic anemia, congenital hypoplastic anemia, pure red cell aplasia, or select cases of secondary thrombocytopenia in a subject; comprising administering to the subject in need of such treatment a therapeutically effective amount of the formulation according to one of the embodiments E1 to E11 and E14 to E19.
E26. A method of treating trichinosis with neurologic or myocardial involvement or tuberculous meningitis with subarachnoid block or impending block when used concurrently with appropriate antituberculous chemotherapy in a subject; comprising administering to the subject in need of such treatment a therapeutically effective amount of the formulation according to any one of the embodiments E1 to E11 and E14 to E19.
E27. A method of treating leukemia or lymphoma for palliative management in a subject comprising administering to the subject in need of such treatment a therapeutically effective amount of the formulation according to any one of the embodiments E1 to E11 and E14 to E19.
E28. A method of treating multiple sclerosis, cerebral edema associated with primary or metastatic brain tumor, or craniotomy in a subject; comprising administering to the subject in need of such treatment a therapeutically effective amount of the formulation according to any one of the embodiments E1 to E11 and E14 to E19.
E29. A method of treating ophthalmic diseases selected from sympathetic opthalmia, temporal arteritis, uveitis, or ocular inflammatory conditions unresponsive to topical corticosteroids in a subject; comprising administering to the subject in need of such treatment a therapeutically effective amount of the formulation according to any one of the embodiments E1 to E11 and E14 to E19.
E30. A method to induce diuresis or remission of proteinuria in a subject; comprising administering to the subject in need of such treatment a therapeutically effective amount of the formulation according to any one of the embodiments E1 to E11 and E14 to E19.
E31. A method to treat respiratory diseases selected from berylliosis, fulminating or disseminated pulmonary tuberculosis, idiopathic eosinophilic pneumonias, or symptomatic sarcoidosis in a subject; comprising administering to the subject in need of such treatment a therapeutically effective amount of the formulation according to any one of the embodiments E1 to E11 and E14 to 19.
E32. A method to treat rheumatic disorders selected from acute gouty arthritis, acute rheumatic carditis, ankylosing spondylitis, psoriatic arthritis, rheumatoid arthritis, juvenile rheumatoid arthritis, dermatomyositis, polymyositis, or systemic lupus erythematosus in a subject; comprising administering to the subject in need of such treatment a therapeutically effective amount of the formulation according to any one of the embodiments E1 to E11 and E14 to E19.
E33. A method for treating inoperable, recurrent, and metastatic endometrial or renal carcinoma in a subject; comprising administering to the subject in need of such treatment a therapeutically effective amount of the formulation according to any one of the embodiments E1 to E8 and E12 to E19
E34. A method to prevent pregnancy in a subject; comprising administering to the subject in need of such treatment a therapeutically effective amount of the formulation according to any one of the embodiments E1 to E8 and E12 to E1
E35. A formulation as defined in any of the embodiments E1 to E19, for use as a medicament.
E36. A formulation as defined in any of the embodiments E1 to E11 and E14 to E19, for use in a method of treating any of the diseases recited in embodiments E21 to E32.
E37. A formulation as defined in any of the embodiments E1 to E8 and E12 to E19, for use in a method of treating inoperable, recurrent, and metastatic endometrial or renal carcinoma.
E38. A formulation as defined in any of the embodiments E1 to E8 and E12 to E19, for use to prevent pregnancy.
E39. The use of a formulation as defined in any of the embodiments E1 to E11 and E14 to E19 in the manufacture of a medicament for use in treating any of the diseases recited in embodiments E21 to E32.
E40. The use of a formulation as defined in any of the embodiments E1 to E8 and E12 to E19 in the manufacture of a medicament for use in the prevention of pregnancy.
E41. The use of a formulation as defined in any of the embodiments E1 to E8 and E12 to E19 in the manufacture of a medicament for use in treating disease treating inoperable, recurrent, and metastatic endometrial or renal carcinoma.
E42. A formulation for parental use comprising 20 mg/mL methylprednisolone, 0.1165 mg/mL MGPC, 9 mg/mL sodium chloride, and water; and wherein the formulation is essentially free of each of polyethylene glycol and polysorbate.
E43. A formulation for parental use comprising 40 mg/mL methylprednisolone, 0.1165 mg/mL MGPC, 9 mg/mL sodium chloride, and water; and wherein the formulation is essentially free of each of polyethylene glycol and polysorbate.
E44. A formulation for parental use comprising 80 mg/mL methylprednisolone, 0.1165 mg/mL MGPC, 9 mg/mL sodium chloride, and water; and wherein the formulation is essentially free of each of polyethylene glycol and polysorbate.
E45. A formulation for parental use comprising 120-160 mg/mL methylprednisolone, 0.4-0.45 mg/mL MGPC, sodium chloride, and water; and wherein the formulation is essentially free of each of polyethylene glycol and polysorbate.
E46. A formulation for parental use comprising 150 mg/mL medroxyprogesterone, 0.223 mg/mL MGPC, 10 mg/mL sodium chloride, and water; and wherein the formulation is essentially free of each of polyethylene glycol and polysorbate.
E47. A formulation for parental use comprising 400 mg/mL medroxyprogesterone, 0.6-0.7 mg/mL MGPC, 10 mg/mL sodium chloride, and water; and wherein the formulation is essentially free of each of polyethylene glycol and polysorbate.
E48. A formulation according to embodiment E6, further wherein the sodium chloride has a concentration of 5 to 13 mg/mL.
E49. A formulation according to embodiment E6, further wherein the sodium chloride has a concentration of 6 to 11 mg/mL.
E50. A formulation according to embodiment E9, wherein the methylprednisolone acetate has a concentration in the range of 20-180 mg/mL.
E51. A formulation according to embodiment E9, wherein the methylprednisolone acetate has a concentration in the range of 20-170 mg/mL.
E52. A formulation according to embodiments E1, wherein the corticosteroid is methylprednisolone acetate with a concentration of 80-180 mg/mL and the quaternary ammonium compound is myristyl gamma picolinium chloride with a concentration equal to 0.25% to 0.33% of the concentration of the methylprednisolone acetate.
E53. A formulation according to embodiments E1 or E52, wherein the tonicity agent is sodium chloride or potassium chloride.
E54. A formulation according to any of embodiments E1, E52, or E53, wherein the methylprednisolone acetate has a concentration of 80-160 mg/mL.
E55. A formulation according to embodiment E54 wherein the concentration of myristyl gamma picolinium chloride is 0.3% of the concentration of methylprednisolone acetate.
E56. A formulation according to embodiment E1, wherein the corticosteroid is 120 mg/mL methylprednisolone acetate, the quaternary ammonium compound is 0.35 mg/mL myristyl gamma picolinium chloride, and the tonicity agent is 9 mg/mL sodium chloride.
E57. A formulation according to embodiment E1, wherein the corticosteroid is 80 mg/mL methylprednisolone acetate, the quaternary ammonium compound is 0.12 to 0.23 mg/mL myristyl gamma picolinium chloride, and the tonicity agent is 9 mg/mL sodium chloride.
E58. A formulation according to embodiment E1, wherein the corticosteroid is 40 mg/mL methylprednisolone acetate, the quaternary ammonium compound is 0.12 mg/mL myristyl gamma picolinium chloride, and the tonicity agent is 9 mg/mL sodium chloride.

### Definitions

The term “ppm,” as used herein means parts per million by weight.

The term “essentially free of polyethylene glycol and polysorbate” means that polyethylene glycol and polysorbate is not deliberately added to improve the properties of the formulation (e.g. physical stability) and, if present at all, does not exceed trace amounts and preferably is less than 100 ppm.

The term “PEG,” as used herein, means “polyethylene glycol.” Typical preferred ranges of molecular weights for PEG for pharmaceutical use ranges from PEG 200 to PEG 8000. PEG 3350, which has an arrange of molecular weight in the certificate of analysis, i.e., 3015-3685, and is used in all or most commercial formulations of medroxyprogesterone and medroxyprogesterone. PEG 3350 is inclusive of the definition of PEG.

The term “PS,” as used herein, means “polysorbate.” Commercially available polysorbates for pharmaceutical use range in grade from 20, 21, 40, 60, 61, 65, 80, 81, 85, 120. Polysorbate 80 is the preferred PS for use in commercial formulations of medroxyprogesterone and is inclusive of the definition of PS.

The term “MRA” as used herein, means methylprednisolone acetate.

The term “vehicle” as used herein, refers to an aqueous solution containing the excipients (e.g., NaCl, and MGPC), without API.

The term “suspension” as used herein, refers to a formulation after adding an insoluble API to the vehicle.

The term “isotonicity agent” as used herein, refers to an additive in a solution that controls isotonicity. Non-limiting examples of isotonicity agents include sodium chloride, potassium chloride.

The term “tonicity agent” as used herein, refers to an additive that when dissolved in a solution can impact the osmotic pressure of that solution. Non-limiting examples of tonicity agents include sodium chloride and potassium chloride. For example, NaCl (a tonicity agent) at 9 mg/mL affords an isotonic solution. For clarity, all isotonicity agents are tonicity agents.

The vehicle for commercially available Depo-Medrol® sterile aqueous suspension is comprised of PEG 3350 for stabilizing the suspension by controlling flocculation of the API particles, sodium chloride for isotonicity, and MGPC as a wetting agent and preservative.

The vehicle for commercially available Depo-Provera© sterile aqueous suspension contains PEG 3350 and PS 80 for stabilizing the suspension, sodium chloride for isotonicity, methylparaben and propylparaben as preservatives.

It has been discovered that by eliminating PEG 3350 and reducing the concentration of myristyl-gamma picolinium chloride (MGPC) found in commercially available dosages, Depo-Medrol® stability is significantly improved in terms of pH drop and resuspendability. Not only does MGPC act as a preservative and wetting agent but also as a cationic surfactant for the controlled flocculation of API particles resulting in excellent redispersibility. Similarly, for Depo-Provera®, it has been discovered that by removing PEG and PS and using only MGPC and sodium chloride in the vehicle, improves the stability of suspension during shelf life.

It has also been discovered that by eliminating PEG, it is possible to achieve novel, stable high-concentration formulations of methylprednisolone that allow for higher doses to be administered to minimize pain, discomfort associated with higher dosage volumes and multiple injections.

### Example 1: Commercial Depo-Medrol® Compared to PEG-Free Formulation

Formulation of a suspension follows a standard two-step process wherein a vehicle is prepared, followed by adding methylprednisolone acetate (MRA) powderto make the suspension, wetting, agitation, high shear mixing, and finally agitation again. Desired amounts of non-API ingredients were sequentially added to Milli-Q water as the temperature was controlled at 23-30° C. The vehicle is filtered through 0.22 μm filters and its pH was readjusted to 6.8-7.0. MRA powder was subsequently weighed out and added to the vehicle, then agitated for 10 minutes. The resulting slurry was subjected to a period of high shear mixing for one minute followed by another ten-minute period of agitation. Finally, the pH of the suspension was adjusted, if needed, to 6.8-7.0 using diluted NaOH or HCl. The suspension temperature was monitored to ensure it remains within the 23-30° C. range.

Two Depo-Medrol® 40 mg/mL suspensions were prepared to compare the characteristics of the product formulated using the current vehicle versus that with a PEG-free vehicle. In the first experiment (T1), the control vehicle which included PEG 3350 (30 mg/mL), MGPC (0.233 mg/mL), and NaCl (9.0 mg/mL) was prepared using the procedure described above. In the second experiment (T2), the vehicle was comprised of 0.1165 mg/mL MGPC and 9.0 mg/mL NaCl. The same MRA batch was used for both T1 and T2 formulations to give a 40 mg/mL API concentration. The settled drug heights (SDH), a marker for resuspendability, after 1 day of settling were 26% and 45% for the control and PEG free suspensions, respectively. Both suspensions required only 2-3 inversions to fully resuspend the API.

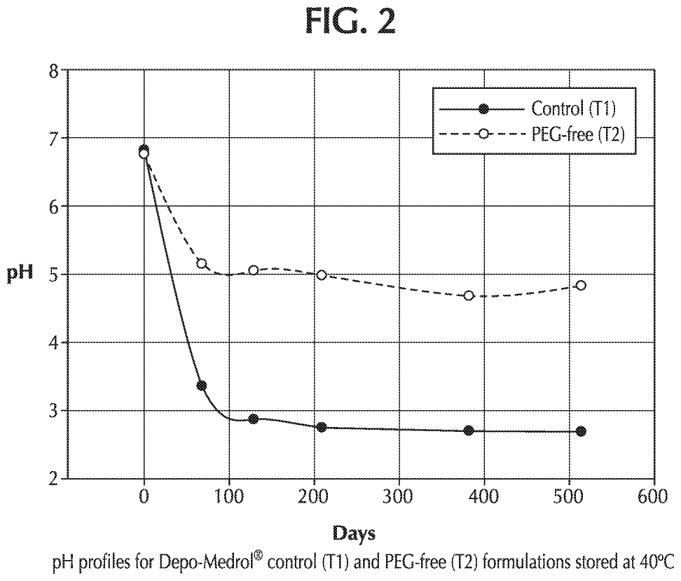

A stability study was conducted in which 1-mL vials were filled with each formulation and the vials were capped with stoppers. The vials were stored in a lab oven in the upright positions at 40° C. Vials were removed at different intervals and the pH was measured. As presented in Table 1 and shown in FIG. 2, the pH for the PEG-free stability samples (T2) has levelled off at about 4.8 after 514 days of storage at 40° C. compared to a pH of 2.7 for the control (T1). The control (T1) required 4 inversions to fully resuspend the API compared to 2 inversions for the PEG-free formulation (T2).

TABLE 1
pH of Depo-Medrol ® control formulation (T1) and
PEG-free formulation (T2) at 40° C.

| Time, days | pH control (T1) | pH PEG-Free (T2) |
|---|---|---|
| 0 | 6.82 | 6.76 |
| 68 | 3.36 | 5.15 |
| 129 | 2.87 | 5.05 |
| 209 | 2.75 | 4.98 |
| 382 | 2.70 | 4.68 |
| 514 | 2.69 | 4.83 |

### Example 2: Commercial Depo-Provera® Suspension Compared to PEG-Free/PS-Free Formulation

Two Depo-Provera® formulations were prepared to compare suspensions formulated using the current vehicle with suspensions formulated with a vehicle prepared using MGPC and NaCl only. The control vehicle contained 1.554 mg/mL methylparaben, 0.17 mg/mL propylparaben, 9.9 mg/mL NaCl, 32.85 mg/mL PEG 3350, and 2.735 mg/mL PS 80. The new vehicle comprised 9.9 mg/mL NaCl and 0.223 mg/mL MGPC. The vehicles were filtered through 0.22μ filter and the filtrate pH was adjusted to 6.4-6.6. Medroxyprogesterone acetate (MPA) powder was subsequently weighed out, added to the vehicle to give a 150 mg/mL API concentration, and agitated for 10 minutes. The resulting slurry was subjected to a period of high shear mixing for one minute followed by another thirty-minute period of agitation. Finally, the pH of the suspension is adjusted, if needed, to 6.4-6.6 using diluted NaOH or HCl. The suspension temperature was monitored to ensure it remains within the 23-30° C. range.

The settled drug heights (SDH) after 1 day of settling were 69% and 95% for the control and PEG-free suspensions, respectively. The control required 5 inversions to fully resuspend the API while the PEG-free suspension required 2 inversions.

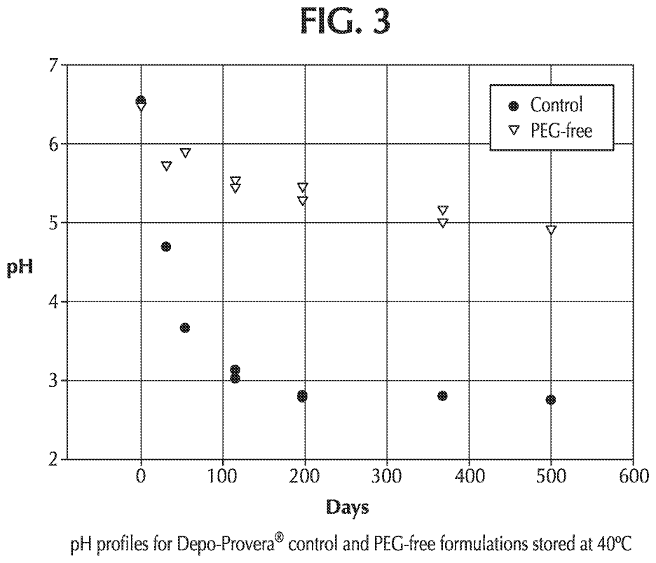

A stability study was conducted in which 1-mL vails were filled with each formulation and the vials were capped with stoppers. The vials were stored in a lab oven in the upright positions at 40° C. Vials were removed at different intervals and the pH was measured. As presented in Table 2 and shown in FIG. 3, the pH for the PEG-free stability samples has levelled off at about 4.9 after 500 days of storage at 40° C. compared to a pH of 2.7 for the control. Further, resuspending the API could not be achieved for the control even after 100 inversions. For the PEG-free formulation, on the other hand, only 8 inversions were required to fully resuspend the API.

TABLE 2
pH of Depo-Provera ® control formulation and
PEG-free formulation at 40° C.

| Time, days | pH control (T1) | pH PEG-Free (T2) |
|---|---|---|
| 0 | 6.54 | 6.48 |
| 31 | 4.69 | 5.73 |
| 54 | 3.66 | 5.90 |
| 115 | 3.02 | 5.54 |
| 115 | 3.13 | 5.45 |
| 197 | 2.81 | 5.46 |
| 197 | 2.78 | 5.29 |
| 368 | 2.80 | 5.01 |
| 368 | 2.80 | 5.17 |
| 500 | 2.75 | 4.92 |

### Example 3: Preparation of High Concentration PEG-Free 120 mg/mL MRA Suspensions

Formulation of a high concentration suspension followed the standard two-step process outlined for the PEG-free composition of Example 1 wherein a vehicle was prepared, followed by adding methylprednisolone acetate (MRA) powder to make the suspension, wetting, agitation, high shear mixing, and finally agitation again. Desired amounts of non-API ingredients were sequentially added to purified water as the temperature was controlled at 23-30° C. The vehicle was filtered through 0.22 μm filters and adjusted to pH 6.0-7.0. MRA powder was subsequently weighed out and added to the vehicle, then agitated for 10 minutes. The resulting slurry was subjected to a period of high shear mixing for one minute followed by another ten-minute period of agitation. Finally, the pH of the suspension was adjusted, if needed, to 6.5-7.0 using diluted NaOH or HCl. The suspension temperature was monitored to ensure it remains within the 23-30° C. range.

### Example 3A: Sedimentation of PEG-Free 120 mg/mL MRA Suspensions

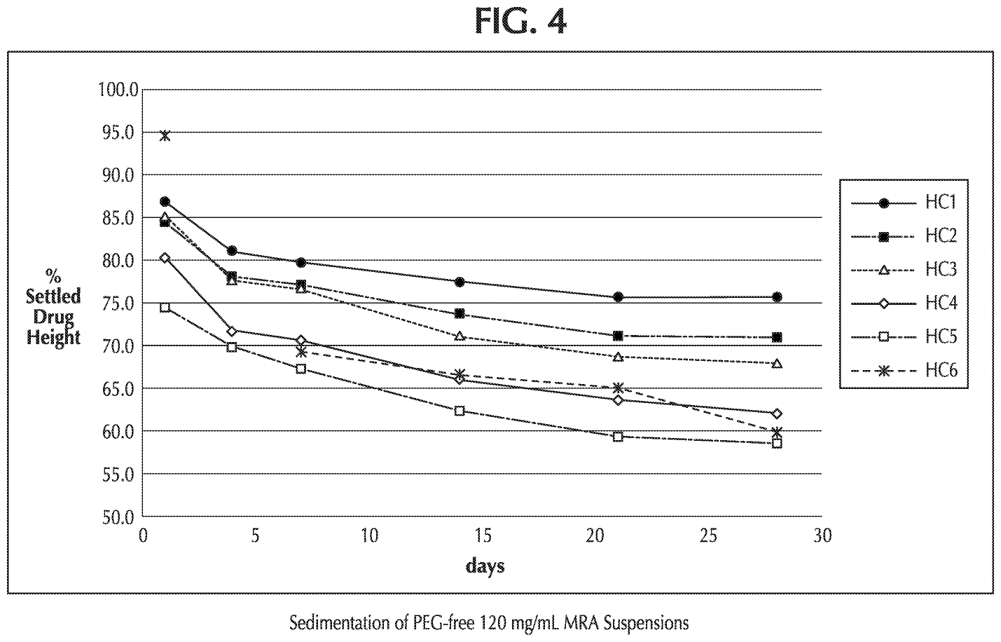

The above procedure was used to compound 120 mg/mL MRA suspensions HC-1 through HC-6 which varied in MGPC (0.3 mg/mL to 0.699 mg/mL). These suspensions were filled into scintillation vials, sealed, and left to stand without agitation to examine solids sedimentation (% Settled Drug Height, SDH). Sedimentation was determined on undisturbed suspensions for up to 30 days. MGPC concentration displayed an impact on both sedimentation rate as well as final stabilized sediment height. Higher levels of MGPC were directly correlated to faster dropping of SDH, as demonstrated in FIG. 4. Concurrently, increasing levels of MGPC were also inversely proportional to final sedimentation height, where the highest levels of MGPC afforded the most compact sediments. In both of these cases, there was a dose-dependent response vs MGPC level for HCl through HC5. HC6 on the other hand, with the highest MGPC concentration, displayed the largest SDH height after 1 day, but achieved a similar final state to that of HC5. FIG. 4 shows that these high concentration PEG free formulations do not suffer from either caking or deflocculation.

### Example 3B: Resuspendability of PEG-Free 120 mg/mL MRA Suspensions

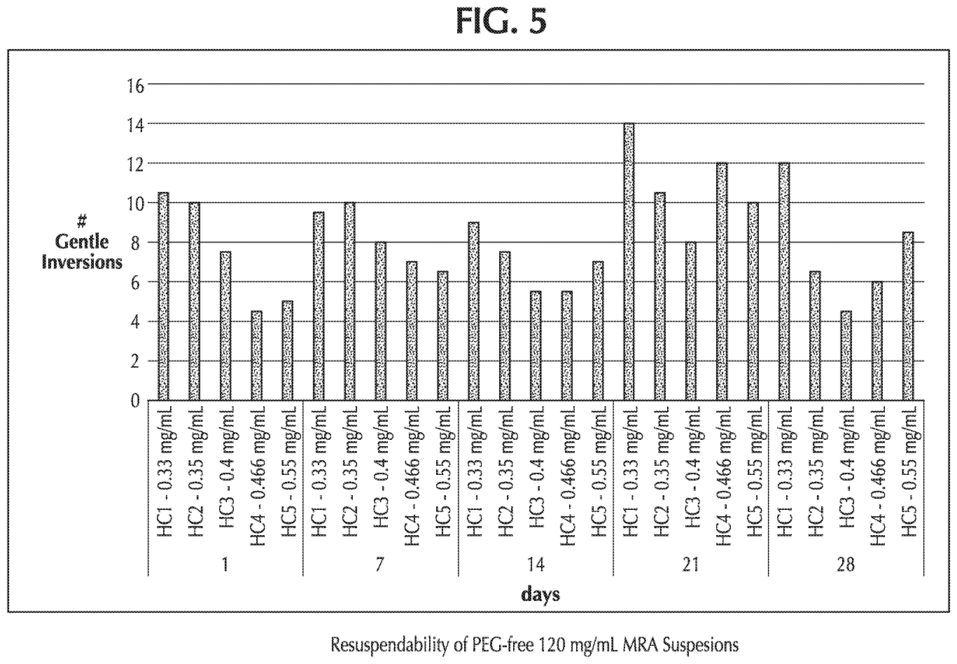

A resuspendability challenge for HC1-HC5 was performed. Separate scintillation vials were filled and sealed. At various timepoints the systems were gently mixed, performed by gentle inversions to achieve fully homogenous dispersions. FIG. 5 plots the average inversions (n=2) to fully resuspend 120 mg/mL MRA at various MGPC concentrations. Between each challenge, a clear, colorless supernatant free of visible particulate was observed. This suspension demonstrated repeatable sedimentation after consistent resuspension challenge and returned to a consistent solids volume.

### Example 3C: Stability of PEG-Free 120 mg/mL MRA Suspensions

The above procedure was used to test the stability of high concentration formulations using compound suspensions HC7-HC10 comprised of MRA (120 mg/mL), NaCl (9 mg/mL) and myristyl-gamma-picolinium chloride (MGPC, ranging from 0.3-0.4 mg/mL). These suspensions were filled into 1 mL glass vials, stoppered, sealed and placed in stability chambers (inverted) assess performance after storage at accelerated aging (40° C.), intermediate (30° C.), and long-term (25° C.) conditions. Suspensions prepared with MGPC levels below 0.3 mg/mL were not viable, and at MGPC concentrations above 0.4 mg/mL was not advantageous at this level of MRA. The pH of these suspensions follows a similar trend as observed for the PEG-free formulation of Example 1 as seen in Table 3. Samples stored at 40° C. appear to level off at about pH 5.0 after 6 months, compared to approximately pH 5.5 at lower temperature conditions.

TABLE 3
pH of 120 mg/mL PEG-free suspensions at various MGPC levels

| Time | HC-7 (MGPC 0.3 mg/mL) | | | HC-8 (MGPC 0.35 mg/mL) | | | HC-9 (0.375 mg/mL) | | | HC-10 (0.4 mg/mL) | | |
|---|---|---|---|---|---|---|---|---|---|---|---|---|
| (m) | 40° C. | 30° C. | 25° C. | 40° C. | 30° C. | 25° C. | 40° C. | 30° C. | 25° C. | 40° C. | 30° C. | 25° C. |
| 0 | 6.82 | 6.82 | 6.82 | 6.27 | 6.27 | 6.27 | 6.40 | 6.40 | 6.40 | 6.92 | 6.92 | 6.92 |
| 1 | 5.65 | — | — | 5.66 | — | — | 5.50 | — | — | 5.56 | — | — |
| 2 | 5.23 | — | — | 5.28 | — | — | 5.28 | — | — | 5.27 | — | — |
| 3 | 5.17 | 5.64 | 5.89 | 5.23 | 5.63 | 5.54 | 5.18 | 5.59 | 5.68 | 5.17 | 5.68 | 5.86 |
| 6 | 4.94 | 5.40 | 5.58 | 4.89 | 5.30 | 5.49 | 4.85 | 5.33 | 5.56 | 4.86 | 5.35 | 5.60 |
| 9 | — | 5.24 | 5.41 | — | 5.20 | 5.40 | — | 5.18 | 5.41 | — | 5.18 | 5.48 |

### Example 4: Preparation of 160 mg/mL and 200 mg/mL PEG-Free MRA Suspensions

Using the procedure above, a series of batches were prepared following a matrix as outlined in Table 4. This study was conducted to determine the solids limit a suspension could feasibly achieved with the reported MRA/MGPC ratio. For this experiment, a “stable suspension” was defined as meeting all of the pre-established criteria: 1) dispersion must form a flocculated suspension, forming a clear supernatant upon standing, 2) dispersion must resuspend with gentle inversions only demonstrating facile resuspension, and 3) must re-settle affording a clear supernatant after resuspending demonstrating robust sedimentation/resuspension). Batches were placed in scintillation vials and monitored over a two-week period. 160 mg/mL MRA can be formulated at this w/w ratio of MRA/MGPC, however, to go beyond, e.g., at 200 mg/mL additional MGPC is likely required to stabilize the suspended particles.

TABLE 4
Matrix of High Concentration MRA formulations Prepared

| API | | MGPC | | NaCl | |
|---|---|---|---|---|---|
| mg/mL | | mg/mL | | mg/mL | |
| 120 | A | 0.233 | 1 | 7 | i |
| 160 | B | 0.35 | 2 | 9 | ii |
| 200 | C | 0.466 | 3 | 11 | iii |
| | | 0.6 | 4 | | |

TABLE 5
Stability Results of High Concentration MRA Formulations

| | Stable | Run | API | MGPC | NaCl |
|---|---|---|---|---|---|
| Cohort 1 | No | 1 | A | 1 | i |
| | Yes | 2 | A | 1 | iii |
| | Yes | 3 | A | 4 | i |
| | Yes | 4 | A | 4 | iii |
| | No | 5 | C | 1 | i |
| | No | 6 | C | 1 | iii |
| | No | 7 | C | 4 | i |
| | No | 8 | C | 4 | iii |
| | No | 9 | B | 3 | i |
| | Yes | 10 | B | 3 | iii |
| Cohort 2 | No | 11 | C | 4 | ii |
| | No | 12 | B | 2 | iii |
| | Yes | 13 | B | 3 | ii |
| | Yes | 14 | B | 4 | i |
| Cohort 3 | No | 15 | B | 2 | ii |

An attempt was made to make a stable 160 mg/mL MRA suspensions with PEG. Batches were prepared as above, with the inclusion of PEG3350. Employing MGPC concentrations at 0.233 mg/mL and 0.466 mg/mL were unsuccessful in obtaining stable, flocculated suspensions of MRA. These preparations did not settle upon standing and could not be shaken to afford a uniform dispersion.

### Example 5: Effect of Salt Concentration on Resuspendability of 120 mg/mL MRA Suspensions

To examine the role of the salt in high concentration suspensions of MRA, suspensions were prepared using the above process, 120 mg/mL MRA and 0.35 mg/mL MGPC were prepared with NaCl (4.5- and 9 mg/mL) and KCl (4.5- and 9 mg/mL), see Table 6 for results.

TABLE 5
Salt concentration behavior of NaCl vs KCl for 120/0.35 MRA/MGPC suspension

| | | | | | | Clear | |
|---|---|---|---|---|---|---|---|
| Salt | Conc. | | Day | % SDH | Resuspends | Supernatant | Notes |
| NaCl | 9 | mg/mL | 1 | 89.8 | Yes | Yes | Readily resuspends with |
| | | | | | | | single inversion |
| | | | 7 | 83.7 | Yes | Yes | Readily resuspends with |
| | | | | | | | single inversion |
| | 4.5 | mg/mL | 1 | 85.7 | Yes | No | Rapidly settling dispersion, |
| | | | 7 | 77.6 | No | No | Solid cake at bottom of |
| | | | | | | | vial remains after 1 |
| | | | | | | | inversion |
| KCl | 9 | mg/mL | 1 | 86.7 | Yes | Yes | Resuspends with single |
| | | | | | | | inversion |
| | | | 7 | 79.4 | Yes | Yes | Resuspends with single |
| | | | | | | | inversion |
| | 4.5 | mg/mL | 1 | 84.5 | Yes | No | Readily resuspends |

| | | | | |
|---|---|---|---|---|
| 7 | 78.1 | No | No | Solid cake at bottom of |
| | | | | vial after 1 inversion |

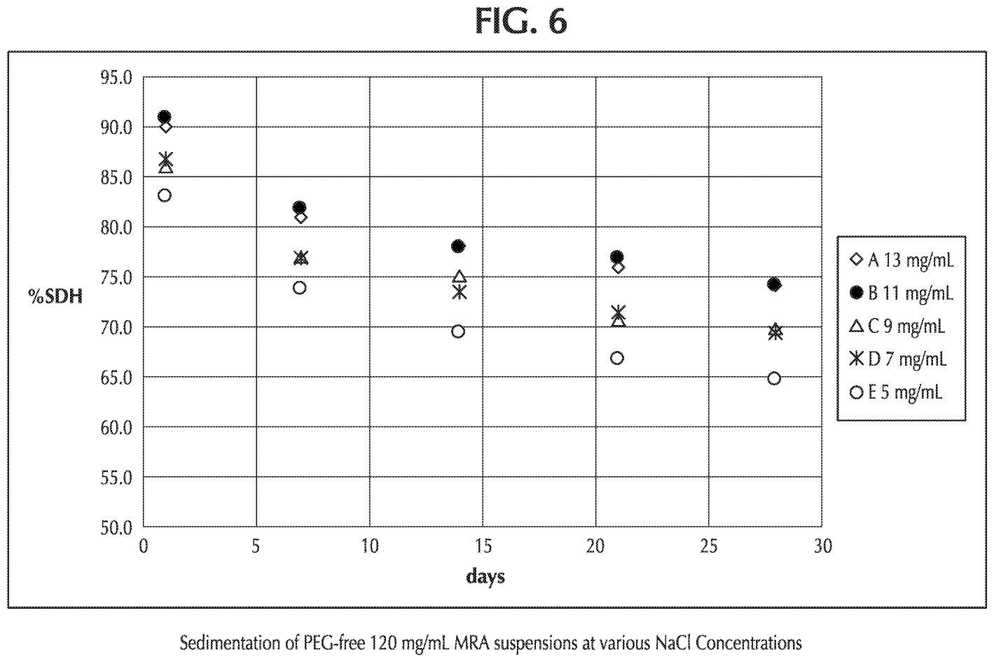

Sodium and potassium salts were able to resuspend 120 mg/mL MRA suspensions with 0.35 mg/mL MGPC at 9 mg/mL concentrations. However, both salt media were readily dispersed throughout the study window. All solutions failed resuspension at 4.5 mg/mL levels of salt, indicating there is a lower limit required for stable dispersions of MRA. In pharmaceutical preparations of parenteral drug products, sodium chloride is preferred as a tonicity agent compared to potassium chloride. A range of formulations were prepared, from 5 mg/mL to 13 mg/mL and % SDH was tracked for 28 day, undisturbed, see FIG. 6. Reducing the salt concentration sees a concomitant compaction of the 120 mg/mL MRA suspension. Interestingly, all levels of sodium chloride from 5 to 13 mg/mL were readily resuspended, mostly in a single inversion (note: 2 inversions were required for 5 mg/mL at 21 days and 28 days, and 2 inversions were required for 13 mg/mL at 28 days). This demonstrates the requirement for ionic composition and a range of 5 to13 mg/mL salt, preferably sodium chloride.

### Example 6: In Vivo Study of 80 mg/mL Depo Medrol® with 120 mg/mL PEG-Free Formulation

A preclinical pharmacokinetic (PK) and pharmacodyamic (PD) study was undertaken to compare 120 mg/mL PEG-fee MRA formulations described herein with the commercially viable 80 mg/mL Depo Medrol®. This was a single-dose, comparative intramuscular study. Dogs were administered 3 mg/kg of either and monitored via in-life phase for 21 days. The 120 mg/mL concentration formulations tested in this study behaved similarly to existing 80 mg/mL (on-market) formula in tolerability, PK, and PD.

We claim:

1. A pharmaceutical aqueous suspension formulation for parental use comprising a corticosteroid, a quaternary ammonium compound, a tonicity agent, and water; and wherein the formulation is essentially free of each of polyethylene glycol and polysorbate.

2. The formulation of claim 1, wherein the corticosteroid is methylprednisolone acetate or medroxyprogesterone acetate.

3. The formulation of claim 2, wherein the quaternary ammonium compound is myristyl gamma picolinium chloride with a concentration of less than 0.5 mg/mL.

4. The formulation of claim 3, wherein the concentration of myristyl gamma picolinium chloride is 0.07-0.3 mg/mL.

5. The formulation of claim 4, wherein the concentration of myristyl gamma picolinium chloride is 0.07-0.23 mg/mL.

6. The formulation of claim 3, wherein the tonicity agent is sodium chloride

7. The formulation of claim 6, further wherein the sodium chloride has a concentration of 9 mg/mL.

8. The formulation of claim 6, wherein the corticosteroid is methylprednisolone acetate.

9. The formulation of claim 8, wherein the methylprednisolone acetate has a concentration in the range of 20-160 mg/mL.

10. The formulation of claim 8, wherein the concentration of methylprednisolone acetate is in the range of 20-80 mg/mL.

11. The formulation of claim 6, wherein the corticosteroid is medroxyprogesterone acetate.

12. The formulation of claim 11, wherein the medroxyprogesterone acetate has a concentration range of 135-165 mg/mL.

13. The pharmaceutical aqueous suspension formulation of claim 1 consisting essentially of methylprednisolone acetate or medroxyprogesterone acetate, myristyl gamma picolinium chloride with a concentration of less than 0.5 mg/mL, sodium chloride, and water.

14. The pharmaceutical aqueous suspension formulation of claim 1 wherein the corticosteroid is methylprednisolone acetate or medroxyprogesterone acetate, the quaternary ammonium compound is myristyl gamma picolinium chloride with a concentration of less than 0.5 mg/mL, and wherein the formulation is maintained at a pH of between 4-7 for a period of at least 100 days at 40° C.

15. The formulation of claim 14 wherein the period is at least 300 days.

16. The formulation of claim 15 wherein the period is at least 500 days.

17. A vial with a headspace containing the formulation according to claim 2, wherein the vial is filled with ambient air in the headspace.

18. The formulation of claim 1 wherein the corticosteroid is methylprednisolone acetate with a concentration of 80-180 mg/mL and the quaternary ammonium compound is myristyl gamma picolinium chloride with a concentration equal to 0.25% to 0.33% of the concentration of the methylprednisolone acetate.

19. The formulation of claim 18 wherein the tonicity agent is sodium chloride or potassium chloride.

20. The formulation of claim 19, wherein the methylprednisolone acetate has a concentration of 80-160 mg/mL.

21. The formulation of claim 20 wherein the concentration of myristyl gamma picolinium chloride is 0.3% of the concentration of methylprednisolone acetate.

22. The formulation of claim 1, wherein the corticosteroid is 120 mg/mL methylprednisolone acetate, the quaternary ammonium compound is 0.35 mg/mL myristyl gamma picolinium chloride, and the tonicity agent is 9 mg/mL sodium chloride.

23. The formulation of claim 1, wherein the corticosteroid is 80 mg/mL methylprednisolone acetate, the quaternary ammonium compound is 0.12 to 0.23 mg/mL myristyl gamma picolinium chloride, and the tonicity agent is 9 mg/mL sodium chloride.

24. The formulation of claim 1, wherein the corticosteroid is 40 mg/mL methylprednisolone acetate, the quaternary ammonium compound is 0.12 mg/mL myristyl gamma picolinium chloride, and the tonicity agent is 9 mg/mL sodium chloride.
